(19) United States
(12) Patent Application Publication
(10) Pub. No.: US 2005/0106686 A1
(43) Pub. Date: May 19, 2005

(54) Process for oxidising dialdehyde polysaccharides

(76) Inventors: Jan Matthijs Jetten, Zeist (NL); Evert Jan Gerrit Timmerman, Woudenberg (NL); Arie Cornelis Besemer, Amerongen (NL); Ronald Tako Marinus Van Den Dool, Culemborg (NL); Johannes Wilhelmus Leonardus Boumans, Ouderkerk Aan De Amstel (NL)

Correspondence Address: YOUNG & THOMPSON, 745 SOUTH 23RD STREET, 2ND FLOOR, ARLINGTON, VA, 22202, US

(21) Appl. No.: 10/494,950

(22) PCT Filed: Nov. 7, 2002
(86) PCT No.: PCT/NL02/00711

(30) Foreign Application Priority Data
Nov. 7, 2001 (EP) 0204268.5

(51) Int. Cl. C12P 19/04; C08B 35/08; C08B 33/08

(52) U.S. Cl. 435/101; 536/123.12; 536/124; 536/56; 536/105

(57) ABSTRACT

Dialdehyde carbohydrates such as dialdehyde starch (DAS) can be oxidised with oxygen or hydrogen peroxide in the presence of a laccase or another enzyme capable of oxidation. The oxidation is mediated by a di-tertiary nitroxyl such as TEMPO. The products contain both aldehyde groups and carboxyl groups and have excellent properties as wet strength agents.

The invention relates to a process of improving the solubility of aldehyde-containing carbohydrates by oxidation of part of the aldehyde groups to carboxylic groups.

WO 00/26257 discloses a process of oxidising dialdehyde starch (DAS) to a monoaldehyde-monocarboxyl starch (MACS) by treatment with a peracid in the presence of a catalytic amount of bromide. The product can be further functionalised, e.g. by reaction with amines, such as aspartic acid. The precursor DAS can be obtained by periodate oxidation of starch.

Although the monoaldehyde-monocarboxyl carbohydrates have interesting properties, the oxidation with peracid and bromide has some drawbacks such as high reasons of cost, the high salt burden resulting from the process, and the suspected toxicity of the chemicals used.

It was found according to the invention that DAS and analogous dialdehyde carbohydrates can be oxidised effectively and without the use of halogens, in such a manner that the aldehyde groups are partially or completely converted to carboxylic groups and thus increase the solubility and versatility of the oxidised carbohydrate, as well as its reactivity because of better accessibility. The oxidation is carried out in the presence of a hydroxylamine or nitroxyl compound, using a chemical reoxidant or an enzyme capable of oxidising hydroxylamines and nitroxyls to nitrosonium ions in the presence of oxygen or hydrogen peroxide. The nitrosonium ion oxidises the aldehyde function to a carboxylic function. The process of the invention is further defined by the characterising features of the appending claims.

The oxidised carbohydrate can be derived from any carbohydrate containing 1,2-dihydroxyethylene groups in its recurring unit, which carbohydrate contains a low level of reducing end groups. Such carbohydrates include non-reducing disaccharides, such as sucrose and trehalose, and oligosaccharides and polysaccharides that are 1,2-, 1,4- or 1,5-linked (pentosans) or 1,2-, 1,4- or 1,6-linked (hexosans). The oligo- and polysaccharides may be of any type, e.g. α-glucans such as starch, starch components (i.e. amylose, amylopectine, dextrins), pullulan (α-1,4, α-1,4, α-1,6-glucan), β-glucans such as cellulose (in particular non-wood), chitin, lichenin etc., furanofructans such as inulin and levan, galactans, arabinogalactans, pentosans (xylans, arabans), (galacto) mannans (guar, locust bean gum), bacterial exopolysaccharides (EPS) and the like and derivatives of such carbohydrates, such as hydrolysates. These oligo- and polysaccharides include heterosaccharides, i.e. those which have different structural units, even if those different units themselves may not have primary hydroxyl groups such as uronic acid units, e.g. in xanthan and carbohydrates derived from algae. The carbohydrates to be oxidised according to the invention include glycosides and other protected carbohydrates. Polysaccharides (degree of polymerisation of more than 10), especially of the glucan types (starch and cellulose) are the preferred carbohydrates.

Modifications of starch, cellulose and other carbohydrates can also be used as starting materials. These comprise partially hydrolysed products, as well as physical and chemical modifications, including hydroxyalkyl, carboxyalkyl and similar derivatives, as well as uronic analogues. The carbohydrates are oxidised to dialdehyde derivatives by (meta) periodate oxidation (see e.g. WO 95/12619), or by any other suitable method, such as methods using manganese oxides. The oxidation may be complete, i.e. the oxidised carbohydrate may exclusively consist of dialdehyde monose units, or the oxidation may be partial, i.e. to a degree of oxidation (dialdehyde monose units) of 0.1-0.99, or e.g. 0.2-0.8.

The dialdehyde carbohydrate thus obtained is oxidised using the process of the invention, involving the use of a nitroxyl compound and a chemical reoxidant or an oxidative enzyme in the presence of oxygen or hydrogen peroxide.

The nitroxyl compound to be used is especially a di-tertiary alkyl nitroxyl compound (or its corresponding hydroxylamine), such as 2,2,6,6-tetramethylpiperidine-N-oxyl (TEMPO). In the following description, reference is made to TEMPO only for the sake of simplicity, but it should be understood that other suitable nitroxyls, i.e. organic nitroxyl compounds lacking α-hydrogen atoms, such as 2,2,5,5-tetramethylpyrrolidine-N-oxyl (PROXYL), 4-hydroxy-, 4-alkoxyl-, 4-acyloxy- and 4-acetamido-TEMPO and derivatives and analogues thereof and those described in WO 95/07303 can be substituted for TEMPO. The nitroxyl may also be immobilised, e.g. by coupling of the hydroxyl group of 4-hydroxy-TEMPO to a suitable carrier, or in the form of a polymeric nitroxyl. The active oxidising species is the nitrosonium ion (oxo-ammonium ion >N⁺═O), that is produced in situ by oxidation of the corresponding hydroxylamine (>N—OH) or nitroxyl radical (>N—O).

The nitroxyl compound is used in a catalytic amount, preferably 0.1-25% by weight, based on the carbohydrate, or 0.1-25 mol % with respect to the carbohydrate. If desired, the reaction can be performed in two steps, the production of the nitrosonium ion being the first and the oxidation of the alcohol function being the second.

The catalysts to be used according to the invention are oxidoreductases or other enzymes that are capable of oxidation in the presence of a suitable redox system. Oxido-reductases, i.e. enzymes capable of oxidation without the presence of further redox systems, to be used in the process of the invention include peroxidases and oxidases, in particular polyphenol oxidases and laccase. Certain hydrolases, such as phytase and lipases, can be used when a firer redox system is present such as a metal complex, e.g. vanadate. Instead of complete enzymes, so-called “synzymes”, i.e. transition metal complexes mimicking enzymes, can be used. Such complexes comprise e.g. vanadium, manganese, iron, cobalt, nickel or copper with complexing agents, in particular polyamines, such as 2,2′-bipyridyl, phenanthroline, tetramethyl-ethylenediamine, penta-methyldiethylenetriamine and their cyclic counterparts such as 1,4,7-trimethyl-1,4,7-tri-azonane, and histidine and its oligomers. The metal-assisted enzymes require hydrogen peroxide, alkyl and ar(alk)yl hydroperoxides (such as tert-butyl hydroperoxide) or chlorite as an ultimate electron acceptor.

Peroxidases (EC 1.11.1.1-1.11.1.11) that can be used according to the invention include the peroxidases which are cofactor-independent, in particular the classical peroxidases (EC 1.11.1.7). Peroxidases can be derived from any source, including plants, bacteria, filamentous and other fingi and yeasts. Examples are horse-radish peroxidase, soy-hull peroxidase, myeloperoxidase, lactoperoxidase, bromo-peroxidase, chloroperoxidase, Arthromyces and Coprinus peroxidases. Several peroxidases are commercially available. The peroxidases require hydrogen peroxide as an electron acceptor.

Polyphenol oxidases (C 1.10.3.1) include tyrosinases and catechol oxidases, such as lignin peroxidase. Suitable polyphenol oxidases may be obtained from fungi, plants or animals. The polyphenol oxidases require oxygen as an electron acceptor. Laccases (EC 1.10.3.2) are sometimes grouped under the polyphenol oxidases, but they can also be classified as a distinct group, sometimes referred to as p-diphenol oxidases. Laccases can be derived from plant sources or from microbial, especially fungal, sources, e.g. of the species Trametes versicolor. The use of recombinant laccases is especially advantageous. The laccases also require oxygen as an electron acceptor.

Alternatively, the oxidation with the nitroxyl compound can be performed with a known chemical capable of reoxidising hydroxylamines to nitroxyls or nitrosonium ions. Such known chemical reoxidants include hypobromite, hypochlorite, or hypochlorite/chlorite mixtures, and peracids such as persulphuric acid. These reoxidants can be used in an amount corresponding to the desired degree of further oxidation. For example, if a 100% dialdehyde starch or dialdehyde cellulose should be further oxidised to an extent that 10% of the aldehyde groups are oxidised to carboxyl groups, 0.1 equivalent (or a slight excess up to e.g. 0.15 equivalent) of sodium hypochlorite or persulphuric acid with respect to the monosaccharide units present can be used.

The process of the invention can be performed under relatively mild conditions, e.g. at a pH between 2 and 10, and at a temperature between 15 and 60° C. (both depending on the particular oxidant, e.g. enzyme or metal complex). The reaction medium can be an aqueous medium, or a homogeneously mixed medium, e.g. of an alcohol/water or an ether/water mixture, or a heterogeneous medium, e.g. a mixture of water and a water-immiscible organic solvent such as a hydrophobic ether, a hydrocarbon or a halogenated hydrocarbon. In the latter case, the enzyme and/or the nitroxyl and the oxidising agent may be present in the aqueous phase and the alcohol substrate and the aldehyde or ketone product may be present in the organic phase. If necessary, a phase transfer catalyst may be used. The reaction medium can also be a solid/liquid mixture, in particular when the enzyme or the nitroxyl is immobilised on a solid carrier. A heterogeneous reaction medium may be advantageous when the substrate or the product is relatively sensitive or when separation of the product from the other reagents may present difficulties.

The oxidation of the dialdehydes results primarily in oxidation of a number of aldehyde groups to carboxylic groups according to the reaction:
R—CHO+>N⁺═O+OH⁻→—COOH+>N—OH
In addition, oxidation of some of the primary hydroxyl groups—if present—may occur according to the reaction:
R′—CH₂OH+>N⁺═O+OH⁻→R′—CHO+>N—OH+H₂O
R′—CHO+>N⁺═O+COH⁻→R′—COOH+>N—OH
Such primary hydroxyl functions will be present e.g. at the C6 position in glucan-type (and fructan, galactan, mannan etc. type) carbohydrates. The oxidation of the primary hydroxyl groups to aldehydes, and partly carboxyls, further enhances the versatility and solubility of the resulting carbohydrate derivatives.

Particularly useful products according to the invention are those with a relatively high aldehyde content, which makes them suitable as wet strength agents and agents capable of further chemical modification, together with a sufficient level of carboxyl groups to make the product water-soluble. In particular, the aldehyde to carboxyl ratio is higher than 1:1. The invention particularly pertains to carbohydrates having an aldehyde to carboxyl content between 4:1 and 49:1, especially between 5:1 and 24:1, wherein all aldehyde groups (free or bound) and carboxyl groups (protonated or ionic) on any position on the oxidised carbohydrate are included. Products having a lower aldehyde to carboxyl ratio are also suitable as wet strength agent and for other purposes, It is preferred that the products of the invention have an aldehyde content of at least 0.8 aldehyde group per recurring monose unit, more preferably between 1.2 and 2.2, while the carboxyl content is preferably between 0.1 and 1.2, most preferably between 0.2 and 0.8.

The products of the invention, especially the oxidised DAS-type (MACS) products, preferably have a water solubility of at least 0.5 g/l (measured at 20° C. at neutral pH). Alternatively, the water solubility of the product is increased with a factor of at least 2, preferably at least 5, with respect to the dialdehyde carbohydrate starting material. The water solubility can be measured e.g. by turbidity measurement using UV-VIS spectrometry.

The products of the invention are very suitable not only as wet strength agents, but also as thickeners, viscosifiers, stabilisers for emulsions and the like, and as starting materials for fierier functionalisation, especially with alcohols, amines, and other agents capable of coupling with an aldehyde function. Such agents include crosslinking agents (diamines, diols and the like), which can be used to crosslink the carbohydrates or to couple them to amino acids, proteins, active groups etc.

The process of the invention can also advantageously be used for modifying biopolymers such as starch or cellulose, to allow further modification (e.g. dyeing of textile, strengthening of textile fibres and anti-pilling) or to adapt viscosity and other physical or chemical properties, for example solubility, emulsifying properties, tackiness, etc. The modified biopolymers can be used as rheology modifiers, e.g. in water-based coating formulations, as reversible crosslinkers e.g. in adhesive formulations, as additives for non-reversible hot melts, for sizing and spinning in textile applications and as moisturisers in personal care applications.

The invention also pertains to derivatives obtained by coupling of the aldehyde carbohydrates described above with e.g. amines, especially by reductive amination, to produce imino or amino derivatives of carbohydrates as defined in the appending claims. Also, the aldehyde carbohydrates can be acetalised with hydroxy-functionalised compounds, e.g. glycolic acid, for further modification.

The products of the invention and the products obtained using the process of the invention can be incorporated into compositions such as wet-strength improving, thickening, viscosifying and/or emulsion-stabilising compositions, optionally together with water and/or other solvents or diluents, fillers, preservatives, further active components, etc. The products of the invention can be contained in such compositions in any amount, e.g. amounts ranging from 0.01 to 99 wt. %, especially 0.5-50 wt. % of the weight of the composition.

### EXAMPLES

### General

Aldehyde contents were determined either by a subtractive method (determining the uronic acid content before and after of oxidation of aldehydes with chlorite and hydrogen peroxide), or by addition of hydroxylamine hydrochloride to produce an oxime and back-titration of liberated hydrochloric acid, or by ¹³C NMR spectroscopy (intensity of C6 signal of aldehyde with respect to C1 of anhydroglucose unit, or intensity of C6 (C═N) in the oxime).

### Example 1

Preparation of Monoaldehyde Monocarboxylic Starch (MACS) using Laccase/TEMPO

1a. Preparation of Dialdehyde Starch (DAS)

122.5 grams (0.76 mole, based on anhydroglucose) of starch (weight corrected for dry matter content) are suspended in 500 ml of demineralised water. The suspension is brought to pH 4.0 and cooled to 5° C. Sodium periodate (179 gram, 0.84 mole; 10% molar excess to starch) is added and the suspension is stirred at 5° C. in the dark for 40 hours. The dialdehyde starch obtained in this fashion is isolated by filtration. The crude product is washed extensively with water until iodate can no longer be detected by reaction with potassium iodide/acid.

1b. Preparation of Monoaldehyde Monocarboxylic Starch (MACS)

The DAS thus prepared was oxidised further using laccase/TEMPO or a derivative of TEMPO, namely 4-acetamido-TEMPO. Twenty grams of DAS were suspended in 1 liter of 50 mM sodium acetate buffer pH 5.15 by means of an ultraturrax. The pH was controlled by means of a pH stat during the entire experiment (0.5 M NaOH). The solution was aerated with oxygen. The oxidation was performed at 38° C. To the suspension of DAS, 4 grains of 4-acetamido-TEMPO and 600 U Trametes versicolor laccase VIIIb (Wacker Chemie) were added. The reaction rate was monitored by means of hydroxide consumption. Three samples were taken varying in the reaction time and the degree of oxidation was determined based on the hydroxide consumption, aldehyde content and carboxylic acid content (see Table 1, methods described above).

TABLE 1
Carboxylic and aldehyde content per monomer unit as determined
using three different assays

| | | Hydroxide | |
|---|---|---|---|
| Sample (reaction | Charge | consumption | Hydroxylamine/HCl |
| time in min) | (mol/mol) | (mol/mol) | titration (mol/mol) |
| 1 (˜220) | 0.13 | 0.13 | 0.14 |
| 2 (˜300) | 0.19 | 0.20 | 0.20 |
| 3 (˜350) | 0.22 | 0.31 | 0.24 |

### Example 2

Measurement of Wet-Strength in Paper Sheets

1. Refining

500-530 grams of Östrand TCF pulp is diluted to 12 litres and refined to 25 SR° by a Laboratory refiner R1L, Escher Wyss. The pulp, when refined, is about 40 g/L so it is diluted to the concentration of 3 g/L.

2. Dynamic Sheet Former, Formette

A wire of 0.4 m² is used and we want the Grammage to be 30 g/m².

The pulp is poured into the beater and the wet strength additive is added during stirring for 5 minutes. If PAE is added, it is added after five minutes and the pulp is stirred again for two minutes. Drum speed 1400 rotations/min, dewatering 30 sec.

3. Press

An absorbent paper is put over the sheet and the wire is taken off. Before pressing another absorbent paper is put over the sheet, so the sheet is between the two absorbent papers. The sheet is pressed at 0.5 bar once. To be able to compare sheets with each other, two sheets of the same composition are made but pressed at two different pressures, 0.5 bar and 5 bar. In this way we can compare them at the same density 400 g/cm³.

4. Drying

The sheet is cut into four pieces and dried two and two with a fixed point for three minutes at a temperature of 140° C.

5. Cutting

The sheet is cut into 15-mm strips for testing of dry and wet strength.

100*100 mm is also cut out for measuring the thickness and Grammage.

6. Conditioning

The strips which wet strength is going to be tested are placed in a heating chamber, 105° C. for 10 minutes. Then both the strips for wet and dry strength are placed in a climate room, temperature 23° C., moisture 50% for four hours.

7. Grammage and Thickness

100*100 mm pieces of the sheet is used for measuring the Grammage and thickness. Grammage is measured on a regular balance on 4 layers to get an average.

Thickness is also measured on four layers and on five different spots to get a good average.

8. Tensile Strength Measurement

The strength both wet and dry is measured in an Inston SCAN-P58:86.

Five 15-mm strips are measured to get an average.

When measuring the wet strength, the strip is soaked for 15 sec in tap water.

### Example 3

Wet and Dry Strength of Sheets with PAE and MAXI as Respective Wet Strength Additive

From sheets prepared as described in Example 2 the wet and dry strength were measured.

The results are given in Table 2.

TABLE 2
Wet strength values (N/m) of sheets containing 5 kg/ton MACS and
10 kg/ton PAE

| | | | Relative |
|---|---|---|---|
| Sample | wet | dry | (%) |
| only PAE, no MACS | 3.3 | 17.9 | 18.5 |
| Sample 1 (see example 1) | 4.7 | 21.1 | 22.5 |
| Sample 2 | 3.8 | 19.5 | 19.6 |
| Sample 3 | 3.7 | 19.0 | 19.6 |
| MACS chemical route | 3.7 | 20.6 | 18.2 |

1-12. (canceled)

13. A process for oxidizing a dialdehyde carbohydrate, comprising subjecting the the dialdehyde carbohydrate to the action of an oxidizing agent in the presence of a nitroxyl compound.

14. The process according to claim 13, wherein the nitroxyl compound is a di-tert-nitroxyl compound.

15. The process according to claim 13, wherein the dialdehyde carbohydrate is oxidized with oxygen or hydrogen peroxide as the oxidizing agent in the presence of an enzyme capable of oxidation and the nitroxyl compound.

16. The process according to claim 15, wherein the enzyme is a polyphenol oxidase or a laccase, and the oxidizing agent is oxygen.

17. The process according to claim 15, wherein the enzyme is a peroxidase, especially horse radish, soy-bean, lignin peroxidase or myelo- or lacto-peroxidase, or halo-peroxidase, and the oxidizing agent is hydrogen peroxide.

18. The process according to claim 13, wherein the carbohydrate is an α-glucan (starch type) or β-glucan (cellulose type) or a derivative thereof.

19. The process according to claim 14, wherein the nitroxyl compound is 2,2,6,6-tetramethylpiperidin-1-oxyl (TEMPO).

20. An oxidized carbohydrate derived from a carbohydrate containing 1,2-dihydroxyethylene groups in its recurring units, at least 20% of the 1,2-dihydroxyethylene groups having been oxidized to dialdehyde groups, and part of the aldehyde groups having been oxidized to carboxylic groups, wherein the ratio of aldehyde groups to carboxylic groups is between 4:1 and 49:1.

21. The oxidized carbohydrate according to claim 20, wherein the ratio of aldehyde groups to carboxylic groups is between 5:1 and 24:1.

22. An oxidized carbohydrate derived from a carbohydrate containing 1,2-dihydroxyethylene groups in its recurring units, at least 20% of the 1,2-dihydroxyethylene groups having been oxidized to dialdehyde groups, and part of the aldehyde groups having been oxidized to carboxylic groups, wherein at least a part of the aldehyde groups has been converted to a group with the formula —CH═N—R or —CH₂—NHR, wherein R is hydrogen, hydroxyl, amino, or a group R¹, OR¹ or NHR¹, in which R¹ is C₁-C₂₀ alkyl, C₁-C₂₀ acyl, a carbohydrate residue, or a group coupled with or capable of coupling with a carbohydrate residue.

23. An oxidized carbohydrate derived from a carbohydrate containing 1,2-dihydroxyethylene groups in its recurring units, at least 20% of the 1,2-dihydroxyethylene groups having been oxidized to dialdehyde groups, and part of the aldehyde groups having been oxidized to carboxylic groups, wherein at least a part of the aldehyde groups has been converted to a group with the formula —CH(OR³)—O—CH₂—COOR² or —CH(—O—CH₂—COOR²)₂, in which R² is hydrogen, a metal cation, an ammonium group or a substituted ammonium group, and R³ is hydrogen or a direct bond to the oxygen atom of a dehydrogenated hydroxyl group of the carbohydrate.

24. A wet-strength improving, thickening, viscosifying and/or emulsion-stabilizing composition comprising an effective amount of an oxidized carbohydrate according to claim 20.

25. A wet-strength improving, thickening, viscosifying and/or emulsion-stabilizing composition comprising an effective amount of an oxidized carbohydrate derivative according to claim 22.

26. A wet-strength improving, thickening, viscosifying and/or emulsion-stabilizing composition comprising an effective amount of an oxidized carbohydrate derivative according to claim 23.
